(19) United States
(12) Patent Application Publication
(10) Pub. No.: US 2021/0275191 A1
(43) Pub. Date: Sep. 9, 2021

(54) CIRCUMFERENTIAL TOURNIQUET WITH MACRO STRAP ADJUSTMENT

(71) Applicant: THE REGENTS OF THE UNIVERSITY OF MICHIGAN, Ann Arbor, MI (US)

(72) Inventors: Kevin Ward, Glen Allen, VA (US); Jeffrey Stephen Plott, Algonac, MI (US)

(21) Appl. No.: 17/193,692

(22) Filed: Mar. 5, 2021

Related U.S. Application Data
(60) Provisional application No. 62/987,004, filed on Mar. 9, 2020.

(51) Int. Cl. A61B 17/132

(52) U.S. Cl. CPC A61B 17/1327; A61B 2017/00367

(57) ABSTRACT

A portable compact tourniquet includes a tourniquet strap and a base operably connected to the tourniquet strap. A strap carriage is operably connected to, and linearly translatable relative to, the base. The strap carriage includes a macro strap adjustment member. A screw assembly is rotatably attached to the base. The screw assembly moves the strap carriage away from the base when the screw assembly is operated in one direction and towards the base when the screw assembly is operated in an opposite direction.

### FIELD OF THE INVENTION

The invention generally relates to tourniquets and more specifically to portable compact circumferential tourniquets with a macro strap adjustment member.

### BACKGROUND OF THE INVENTION

Hemorrhage from vascular injuries in the extremities, such as the arms and legs, can be difficult to treat by a single person. While the treatment of such injuries is challenging when they occur in civilian populations, treatment may be even more difficult in combat situations. Improvements in body armor have reduced mortality from combat injuries to the chest. However, the incidence of injuries to the extremities and the associated mortality rates remain high. Recent efforts have developed better tourniquets for treatment of these extremities wounds.

Controlling hemorrhage by application of direct manual pressure may be particularly challenging in cases where the injured person is alone. In fact, most current tourniquet devices are designed to be applied “one-handed.” Because of this, it can be difficult and very painful to achieve a tourniquet pressure that stops blood flow in the limb because of the narrow width of most tourniquet bands.

### SUMMARY

In accordance with one exemplary aspect of the present invention, a portable compact tourniquet includes a tourniquet strap and a base operably connected to the tourniquet strap. A strap carriage is operably connected to, and linearly translatable relative to, the base. The strap carriage includes a macro strap adjustment member. A screw assembly is rotatably attached to the base. The screw assembly moves the strap carriage away from the base when the screw assembly is operated in one direction and towards the base when the screw assembly is operated in an opposite direction.

In accordance with another exemplary aspect of the present invention a method of applying a portable compact tourniquet includes providing a portable compact tourniquet having a tourniquet strap, a base, a strap carriage, and a screw assembly. The tourniquet strap is attached to the strap carriage. The tourniquet strap is threaded through a first base slot formed in the base. The tourniquet strap is wrapped around an object to be occluded. The tourniquet strap is threaded through a second base slot formed in the base. The tourniquet strap is threaded through a macro strap adjustment member and a free end of the tourniquet strap is pulled until there is no slack in the tourniquet strap.

In further accordance with any one or more of the foregoing aspects, a portable compact tourniquet may further include any one or more of the following preferred forms.

In some preferred forms, the macro strap adjustment member is pivotably attached to the strap carriage.

In other preferred forms, the macro strap adjustment member includes a first slot and a second slot that are parallel to one another.

In yet other preferred forms, the first slot and the second slot are separated by an elongated peg.

In yet other preferred forms, the macro strap adjustment member is pivotably attached to the strap carriage with a pivot pin, and the first slot and the second slot are distal to the pivot pin relative to the screw assembly.

In yet other preferred forms, the tourniquet strap is disposed through the first slot, over the elongated peg, and back through the second slot.

In yet other preferred forms, a screw lock secures the screw assembly in a deployed position.

In yet other preferred forms, the screw lock is a set screw.

In yet other preferred forms, the strap carriage further comprises a strap securing opening opposite the macro strap adjustment member.

In yet other preferred forms, the base includes a curved bottom surface.

In yet other preferred forms, the base further comprises a first base slot and a second base slot opposite the first base slot.

In yet other preferred forms, at least one of the first base slot and the second base slot includes a tapered internal channel.

In yet other preferred forms, the tapered internal channel is wider near the bottom surface of the base than near a top of the base.

In yet other preferred forms, the screw assembly includes a handle disposed at one end of the screw assembly.

In yet other preferred forms, the handle includes two outwardly extending shoulders.

In yet other preferred forms, the screw assembly includes a first screw and a second screw that is nested within the first screw.

In yet other preferred forms, the first screw has a larger diameter than the second screw.

In yet other preferred forms, the first screw has a hollow central bore with female threads, the female threads cooperating with male threads on an outer surface of the second screw.

In yet other preferred forms, the screw assembly is operated to move the strap carriage away from the base, which causes the macro strap adjustment member to move to a locked position.

### BRIEF DESCRIPTION OF THE DRAWINGS

FIG. 1 is side cross-sectional view of a portable compact tourniquet constructed in accordance with the teachings of the disclosure, the portable compact tourniquet having a macro strap adjustment member in an unlocked position.

FIG. 2 is side cross-sectional view of the portable compact tourniquet of FIG. 1, the portable compact tourniquet having the macro strap adjustment member in a locked position.

FIG. 3 is a side perspective view of the portable compact tourniquet of FIG. 1, without a tourniquet strap for ease of viewing.

FIG. 4 is a side view of the portable compact tourniquet of FIG. 3.

FIG. 5 is top plan view of the portable compact tourniquet of FIG. 3.

FIG. 6 is a side perspective view of the portable compact tourniquet of FIG. 2, without a tourniquet strap for ease of viewing.

FIG. 7 is a side view of the portable compact tourniquet of FIG. 6.

FIG. 8 is top plan view of the portable compact tourniquet of FIG. 6.

FIG. 9A is a side cross-sectional view of the portable compact tourniquet of FIG. 2 with a screw lock engaged.

FIG. 9B is a side cross-sectional view of the portable compact tourniquet of FIG. 2 with the screw lock disengaged.

### DETAILED DESCRIPTION

The disclosed tourniquets facilitate blood occlusion in patient limbs in emergency situations while enhancing comfort and intuitive use. A screw mechanism in the disclosed tourniquets is selectively lockable, thus allowing for the tourniquet to hold high pressures with very little user effort to ensure that the supply of blood is interrupted to the wound site. Additionally, the disclosed tourniquets include a self-locking tourniquet strap macro-adjustment mechanism to speed adjustment and application of the tourniquet strap.

The tourniquet strap may comprise a fabric, such as nylon or cotton. Other materials may also be used for the tourniquet strap. The tourniquet strap feeds through a tourniquet mechanism which includes a screw assembly, a pivot housing, and a strap carriage. In one embodiment, the screw assembly includes the tourniquet strap macro-adjustment mechanism, which allows large or gross adjustments to the length of tourniquet strap that is connected to the tourniquet mechanism. The tourniquet strap macro-adjustment mechanism is used for initial placement and sizing of the tourniquet strap around the patient limb. The tourniquet strap macro-adjustment mechanism has a locking feature that secures a desired length of the tourniquet strap when the macro-adjustment is complete.

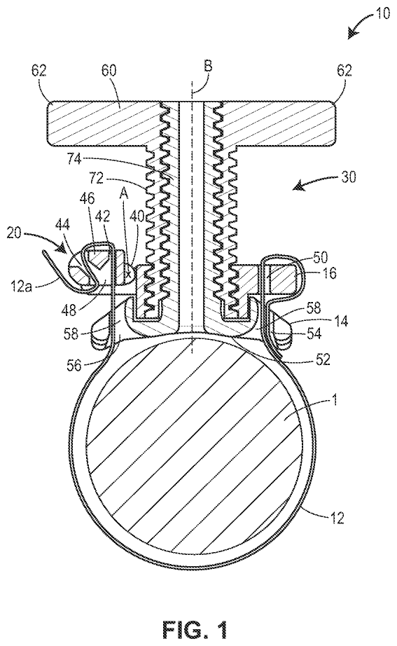

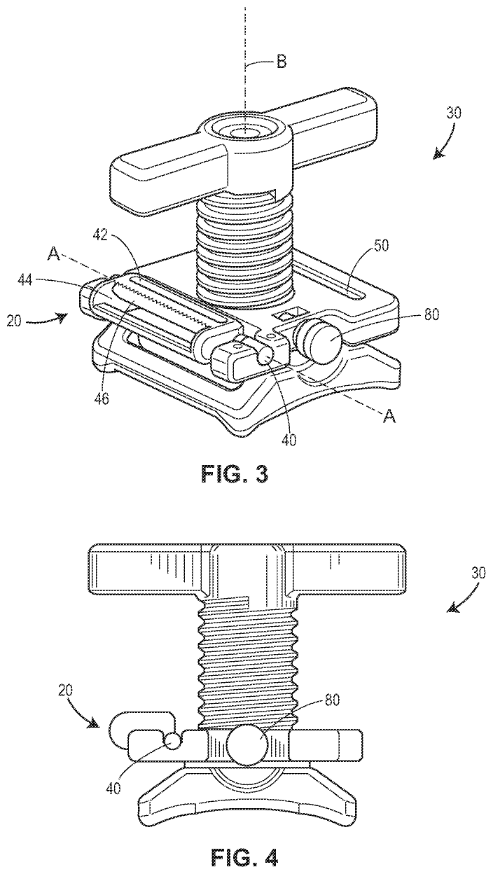

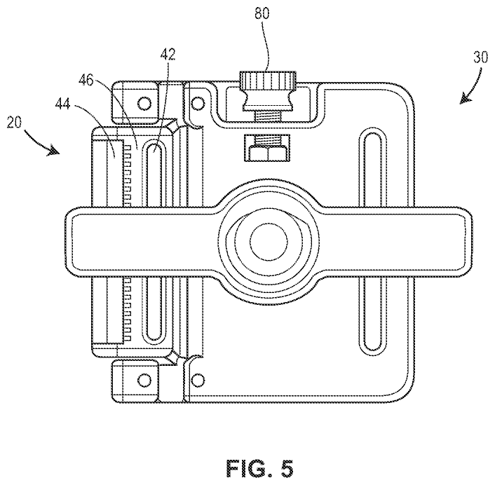

Turning now to the figures, FIG. 1 illustrates one embodiment of a portable compact tourniquet 10 with a macro strap adjustment member 20 in an unlocked position. The unlocked position illustrated in FIG. 1 is used for initial placement and gross tightening of a tourniquet strap 12 around a patient limb. FIGS. 3-5 are additional views of the portable compact tourniquet 10 without the tourniquet strap 12 for ease of viewing.

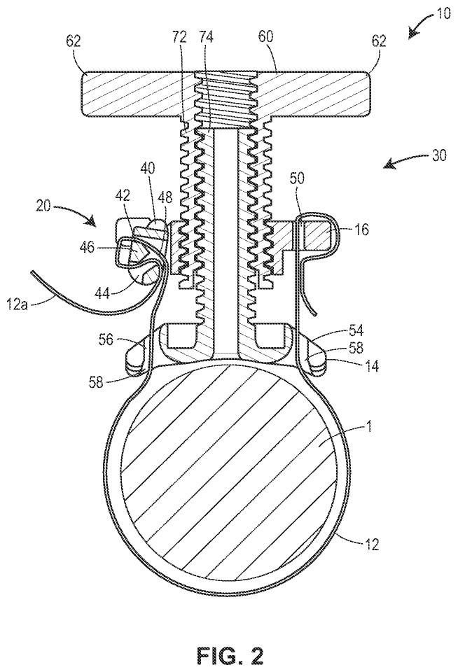

FIG. 2 illustrates the portable compact tourniquet 10 of FIG. 1, with the macro strap adjustment member 20 in a locked position. The locked position illustrated in FIG. 2 is used for occlusion tightening of the tourniquet strap 12. More specifically, the macro adjustment member 20 in the locked position fixes the length of tourniquet strap attached to a strap carriage 16. A screw assembly 30 is then used to move the strap carriage 16 away from a base 14, which applies occlusion pressure to the patient limb 1.

The portable compact tourniquet 10 includes the tourniquet strap 12 and the base 14, which is operably connected to the tourniquet strap 12. The strap carriage 16 is operably connected to, and linearly translatable relative to, the base 14. The strap carriage 16 includes the macro strap adjustment member 20 for making large or gross adjustments to the length of the tourniquet strap 12 attached to the strap carriage 16. The screw assembly 30 is rotatably attached to the base 14. The screw assembly 30 moves the strap carriage 16 away from the base 14 when the screw assembly 30 is operated in one direction, to increase occlusion pressure on the patient limb 1, and towards the base 14 when the screw assembly is 30 operated in an opposite direction, to release occlusion pressure on the patient limb 1.

FIG. 1 illustrates the macro strap adjustment member 20 in the unlocked position, which allows the tourniquet strap 12 to slide freely, thereby facilitating large changes in the length of the tourniquet strap 12 that is looped around the patient limb 1. FIG. 2 illustrates the macro strap adjustment member 20 in a locked position, which prevents further sliding of the tourniquet strap 12 through the macro strap adjustment member 20, thereby fixing the length of the tourniquet strap 12 looped around the patient limb 1. When the macro strap adjustment member 20 is in the locked position of FIG. 2, the screw assembly 30 may be actuated to increase circumferential pressure (or occlusion pressure) around the patient limb 1 to occlude blood flow distal to the tourniquet strap 12.

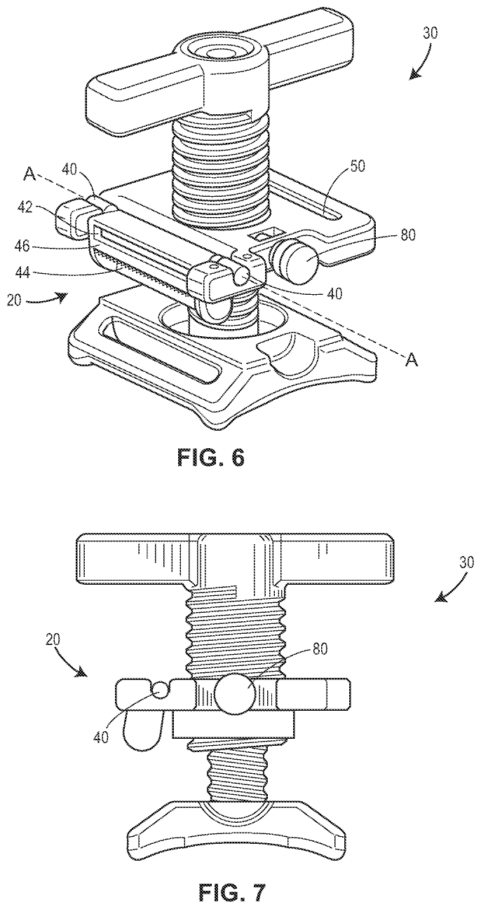

The macro strap adjustment member 20 is pivotably attached to the strap carriage 16. The macro strap adjustment member 20, in one embodiment, may be pivotably attached to the strap carriage with a hinge, such as a pivot pin 40, that pivots about a pivot axis A (best viewed in FIGS. 3 and 6). The pivot axis A may be substantially perpendicular to a screw axis B of the screw assembly 30. In the drawings of FIGS. 1 and 2, the pivot axis A is also perpendicular to the plane of the figure.

The macro strap adjustment member 20 includes a first slot 42 and a second slot 44 that extend parallel to the pivot axis A. The first slot 42 and the second slot 44 are separated by an elongated peg 46. The elongated peg 46 extends parallel to the pivot axis A. The elongated peg 46 may have a cross sectional shape of an irregular pentagon, such as an isosceles right pentagon, similar to the shape of a home plate in baseball, as illustrated best in FIGS. 1 and 2. The first slot 42 and the second slot 44 are arranged to allow the tourniquet strap 12 to loop around the elongated peg 46 and back out a common bottom slot 48. In other words, the tourniquet strap 12 is passed upward, away from the base 14, through the first slot 42, around the elongated peg 46, and downward, towards the base 14, through the second slot 44 and out the bottom slot 48.

In one embodiment, the first slot 42 and the second slot 44 are distal to, or outboard of, the pivot pin 40, relative to the screw assembly 30. By being distal to the pivot pin 40, when the tourniquet strap 12 is pulled tight, the tourniquet strap 12 creates a moment by pulling on the elongated peg 46, which causes the macro strap adjustment member 20 to pivot about the pivot axis A from the unlocked position (FIG. 1) to the locked position (FIG. 2). For example, as long as the free end 12a of the tourniquet strap 12 is pulled upward, away from the base 14, as illustrated in FIG. 1, the macro strap adjustment member 20 remains in the unlocked position to facilitate macro adjustments of the tourniquet strap 12. Once the macro adjustments are complete, the free end 12a of the tourniquet strap is pulled downward, towards the base 14, which causes the macro strap adjustment member 20 to pivot to the closed position (FIG. 2), thereby naturally locking the tourniquet strap 12 in the macro adjusted position by pinching the tourniquet strap 12 between the macro strap adjustment member 20 and a portion of the strap carriage 16.

On the side opposite the macro strap adjustment member 20, the strap carriage 16 includes a strap securing opening 50. The strap securing opening 50 is a slot that extends parallel to the pivot axis A. The strap securing opening 50 allows a loop of the tourniquet strap 12 to be secured to the strap carriage 16 while leaving the free end 12a of the tourniquet strap 12 unattached to be threaded through the macro strap adjustment member 20, as described above.

The base 14 may include a curved bottom surface 52. The curved bottom surface 52 may have a radius of curvature similar to that of a human arm or leg to maximize contact with the patient limb, thereby enhancing circumferential pressure when the tourniquet strap 12 is tightened.

A first base slot 54 and a second base slot 56 are formed in the base 14 to guide and retain the tourniquet strap 12. The first base slot 54 is formed opposite the second base slot 56 on the base 14. At least one of the first base slot 54 and the second base slot 56 includes a tapered internal channel 58. The tapered internal channel 58 is wider near the curved bottom surface 52 of the base 14 than near a top of the base 14.

The screw assembly 30 includes a handle 60 disposed at one end of the screw assembly 30. The handle 60 provides leverage for an operator to operate the screw assembly 30. In some embodiments, the handle 60 includes two outwardly extending shoulders 62. In other embodiments, the handle 60 may have a different configuration, or another structure may be substituted for the handle 60, for example a round or other shaped handle 60 may be used.

The screw assembly 30 includes a first screw 72 and a second screw 74 that is nested within the first screw 72. The first screw 72 has a larger diameter than the second screw 74. The first screw 72 has a hollow central bore with female threads, the female threads cooperating with male threads on an outer surface of the second screw 74.

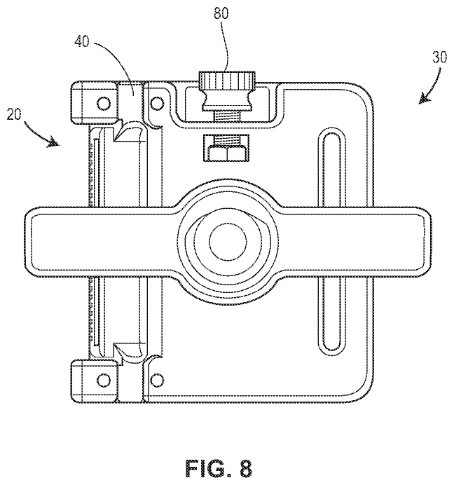

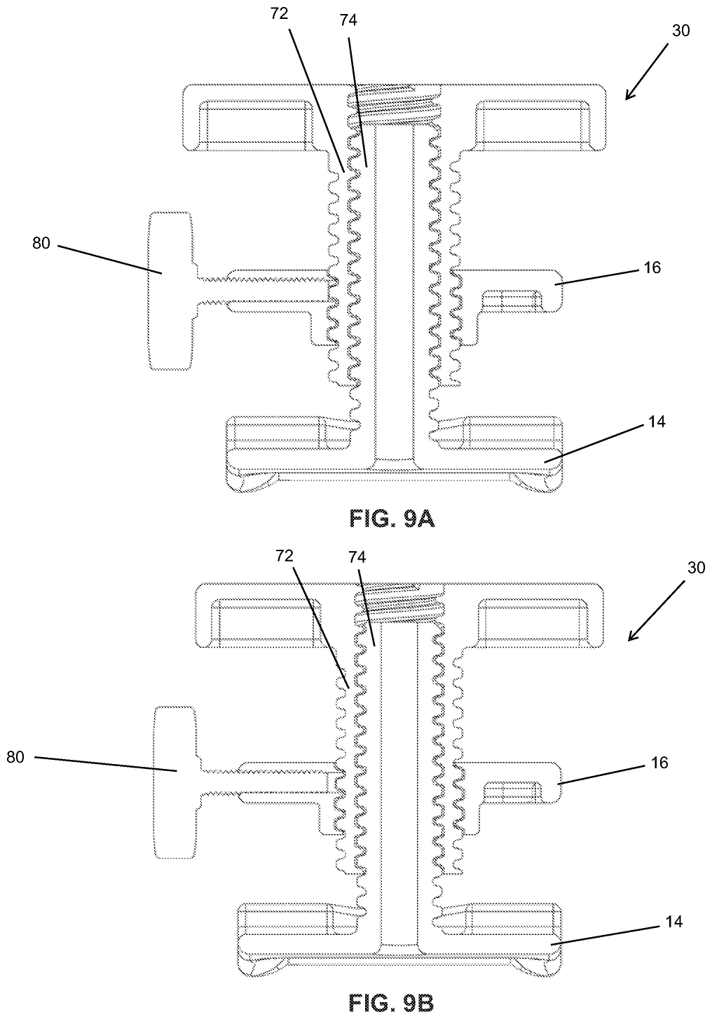

A screw lock 80 (FIGS. 3-9) secures the screw assembly 30 in a deployed position (i.e., FIG. 2, FIG. 9A, and FIG. 9B), where the strap carriage 16 is spaced from the base 14. FIG. 9A illustrates the screw lock 80 engaged and FIG. 9B illustrates the screw lock 80 disengaged. In some embodiments, the screw lock 80 is a set screw. A user may engage the screw lock 80 when the screw assembly 30 is in the deployed position to prevent the screw assembly 30 from reversing due to the tension on the tourniquet strap 12 and/or from reversing when unintentional force (such as a bump) is applied to the handle 60. The screw lock 80 also prevents screw assembly 30 reversal from other unintentional outside forces, such as vibration. In some embodiments, the screw lock 80 increases the frictional force between screw threads to prevent unintentional screw assembly 30 reversal. By preventing unintentional screw assembly 30 reversal, the screw lock 80 also prevents unintentional translation of the strap carriage 16 relative to the base 14.

A method of applying the portable compact tourniquet 10 includes providing the portable compact tourniquet 10 which includes the tourniquet strap 12, the base 14, the strap carriage 16, and the screw assembly 30, as described above. The tourniquet strap 12 is attached to the strap carriage 16 at the strap securing opening 50. The free end 12a of the tourniquet strap 12 is threaded through the first base slot 54 formed in the base 14. The free end 12a of the tourniquet strap 12 is wrapped around an object to be occluded, such as a patient limb 1, and then is threaded through the second base slot 56 formed in the base 14. The free end 12a of the tourniquet strap 12 is threaded through the macro strap adjustment member 20, as described above and the free end 12a of the tourniquet strap 12 is pulled upwards, away from the base 14, until there is no slack in the tourniquet strap 12. The free end 12a of the tourniquet strap 12 is then pulled downward, towards the base 14, to engage the locking feature of the macro strap adjustment member 20 by pivoting the macro strap adjustment member 20 to the locked position (FIG. 2).

After the macro strap adjustment member 20 is locked, the screw assembly 30 is operated, by turning the handle 60, to move the strap carriage 16 away from the base 14, which also causes the macro strap adjustment member 20 to move to a locked position if not already in the locked position due to pulling on the tourniquet strap 12.

In one embodiment, the screw assembly 30 is sized to provide approximately 1.75-2.00 inches of travel for the strap carriage 16, which is enough vertical travel to ensure that occlusion pressure is reached under all conditions.

If increased torque is required, a larger handle may be used to provide more leverage. In addition to the screw lock, in another embodiment, the disclosed compact tourniquets advantageously maintain tightening force on the tourniquets strap so that a user doesn't need to constantly apply the torque to hold the pressure. More specifically, the screw assembly 30, in some embodiments, may include a locking feature, such as a ratchet, which prevents the screw assembly from turning in a loosening direction unless the lock is released. This also allows fine-tuning of the occlusion pressure through tightening at any incremental turning of the screw.

In other embodiments, a writing surface may be provided for recording a time that the tourniquet is applied, which can be important information for a doctor or other medical person to know when evaluating treatment options.

The disclosed compact tourniquets are easy to use, generate great mechanical advantage in tightening, and utilize wide tourniquet bands for less pain and more effective occluding pressures during application. The disclosed tourniquets are also easily deployable and operable with one hand while operating solely on mechanical power generated by the user, so that a source of electrical power is not needed. Furthermore, due to the compact nature the disclosed tourniquets are easily portable and generally light weight so that they may be deployed almost anywhere. Finally, the macro strap adjustment member facilitates rapid sizing of the tourniquet strap, which results occlusion pressure being applied more quickly, thereby minimizing blood loss.

While the present invention has been described with respect to a particular embodiment of the present invention, this is by way of illustration for purposes of disclosure rather than to confine the invention to any specific arrangement as there are various alterations, changes, deviations, eliminations, substitutions, omissions and departures which may be made in the particular embodiment shown and described without departing from the scope of the claims.

What is claimed is:

1. A portable compact tourniquet comprising:
a tourniquet strap;
a base operably connected to the tourniquet strap;
a strap carriage that is linearly translatable relative to the base, the strap carriage including a macro strap adjustment member; and
a screw assembly that is rotatably attached to the base,
wherein the screw assembly moves the strap carriage away from the base when the screw assembly is operated in one direction and towards the base when the screw assembly is operated in an opposite direction.

2. The portable compact tourniquet of claim 1, wherein the macro strap adjustment member is pivotably attached to the strap carriage.

3. The portable compact tourniquet of claim 2, wherein the macro strap adjustment member includes a first slot and a second slot that are parallel to one another.

4. The portable compact tourniquet of claim 3, wherein the first slot and the second slot are separated by an elongated peg.

5. The portable compact tourniquet of claim 4, wherein the macro strap adjustment member is pivotably attached to the strap carriage with a pivot pin and the first slot and the second slot are distal to the pivot pin relative to the screw assembly.

6. The portable compact tourniquet of claim 4, wherein the tourniquet strap is disposed through the first slot, over the elongated peg, and back through the second slot.

7. The portable compact tourniquet of claim 1, further comprising a screw lock that secures the screw assembly in a deployed position.

8. The portable compact tourniquet of claim 7, wherein the screw lock is a set screw.

9. The portable compact tourniquet of claim 1, wherein the strap carriage further comprises a strap securing opening opposite the macro strap adjustment member.

10. The portable compact tourniquet of claim 1, wherein the base includes a curved bottom surface.

11. The portable compact tourniquet of claim 10, wherein the base further comprises a first base slot and a second base slot opposite the first base slot.

12. The portable compact tourniquet of claim 11, wherein at least one of the first base slot and the second base slot includes a tapered internal channel.

13. The portable compact tourniquet of claim 12, wherein the tapered internal channel is wider near the curved bottom surface of the base than near a top of the base.

14. The portable compact tourniquet of claim 1, wherein the screw assembly includes a handle disposed at one end of the screw assembly.

15. The portable compact tourniquet of claim 1, wherein the screw assembly includes a first screw and a second screw that is nested within the first screw.

16. The portable compact tourniquet of claim 15, wherein the first screw has a larger diameter than the second screw.

17. The portable compact tourniquet of claim 16, wherein the first screw has a hollow central bore with female threads, the female threads cooperating with male threads on an outer surface of the second screw.

18. A method of applying a portable compact tourniquet, the method comprising:
providing a portable compact tourniquet including a tourniquet strap, a base, a strap carriage, and a screw assembly;
attaching the tourniquet strap to the strap carriage;
threading the tourniquet strap through a first base slot formed in the base;
wrapping the tourniquet strap around an object to be occluded;
threading the tourniquet strap though a second base slot formed in the base;
threading the tourniquet strap through a macro strap adjustment member; and
pulling a free end of the tourniquet strap until there is no slack in the tourniquet strap.

19. The method of claim 18, further comprising:
operating the screw assembly to move the strap carriage away from the base, which causes the macro strap adjustment member to move to a locked position.

20. A portable compact tourniquet comprising:
a tourniquet strap;
a base operably connected to the tourniquet strap;
a strap carriage that is linearly translatable relative to the base;
a screw assembly that is rotatably attached to the base; and
a screw lock that secures the screw assembly in a deployed position and prevents unintentional movement of the screw assembly,
wherein the screw assembly moves the strap carriage away from the base when the screw assembly is operated in one direction and towards the base when the screw assembly is operated in an opposite direction.
